(19) United States
(12) Patent Application Publication
(10) Pub. No.: US 2025/0102495 A1
(43) Pub. Date: Mar. 27, 2025

(54) LOT RELEASE ASSAYS FOR IGF-1/IGFBP COMPLEXES

(71) Applicant: Oak Hill Bio Ltd, Altrincham (GB)

(72) Inventors: Craig KAFTAN, Lexington, MA (US); Riddhi DALVI, Lexington, MA (US)

(21) Appl. No.: 18/832,155

(22) PCT Filed: Jan. 23, 2023
(86) PCT No.: PCT/EP2023/051510

§ 371 Date: Jul. 23, 2024

Related U.S. Application Data
(60) Provisional application No. 63/302,447, filed on Jan. 24, 2022.

(51) Int. Cl. G01N 33/50; C07K 14/47; C07K 14/65; G01N 33/68

(52) U.S. Cl. CPC G01N 33/5044; C07K 14/4743; C07K 14/65; G01N 33/6893; G01N 2333/4745; G01N 2333/65

(57) ABSTRACT

The present invention provides an accurate and reliable lot release assay to, inter alia, assess the potency of compositions comprising IGF-1/IGFBP complexes for protein replacement therapy, including a serial dilution of an aliquot of complex to generate a series of diluted samples comprising the rhIGF-1/rhIGFBP complex and contacting said diluted samples with osteosarcoma cells for a suitable period.

### BACKGROUND

Insulin-like growth factor 1 (IGF-1) is a small protein hormone that is essential for normal growth and development. While IGF-1 deficiency is most commonly associated with short statue, severe IGF-1 deficiency can lead to a wide array of life threatening diseases and conditions. Premature infants are particularly vulnerable to severe IGF-1 deficiency and have heightened risk of developing a host of conditions.

Of an estimated 4.2 million live births in the United States each year, approximately 383,000 (about 9%) occur prematurely. Preterm labor and its complications are major perinatal public health issues in developed societies today. Low birth-weight infants or infants born prematurely miss a major part of the critical period of in utero growth. They account for half of all infant deaths and three-quarters of long-term morbidity. Their care results in a heavy burden on the national economy, because of the high costs of special care in both the neonatal period and over the life-span of survivors. Many survivors also have diminished quality of life because of physical complications resulting directly from prematurity.

The lungs, digestive system, and nervous system (including the brain) are not fully developed in premature babies and are particularly vulnerable to complications. Common medical problems encountered in preterm infants including, for example, retinopathy of prematurity, developmental delay, mental retardation, Chronic Lung Disease (CLD), Bronchopulmonary Dysplasia (BPD), Pulmonary Hypertension (PH), Right ventricular hypertrophy (RVH), Necrotizing Enterocolitis (NEC), and Intraventricular Hemorrhage (IVH).

Treatment and/or prevention of these and other conditions resulting from a deficiency of IGF-1 require accurate and reproducible dosages of replacement IGF-1. This means the quality and/or activity of the protein needs to be quantifiable and reproducible. Thus, it is necessary to test manufactured recombinant protein.

### SUMMARY

The present invention provides an accurate and reliable lot release assay to, inter alia, assess the potency of compositions comprising rhIGF-1/rhIGFBP-3 complexes for protein replacement therapy. The present invention is based on the surprisingly robust and consistent ability of the disclosed lot release assay to measure bioactivity of the composition comprising rhIGF-1/rhIGFBP-3 and control for sample variability, thereby providing a more accurate and reliable assessment of potency.

The disclosure is summarized by the following numbered paragraphs:
1. A lot release assay comprising:
   (a) providing a lot sample comprising a recombinantly produced human IGF-1/human IGF-1 Binding Protein (rhIGF-1/rhIGFBP) complex;
   (b) preparing a serial dilution from the lot sample in step (a) to generate a series of diluted samples comprising the rhIGF-1/rhIGFBP complex;
   (c) contacting cells (for example osteosarcoma cells) with each of the diluted samples from step (b) for a predetermined period;
   (d) determining the activity of the rhIGF-1/rhIGFBP complex in the osteosarcoma cells in each of the diluted samples;
   (e) generating a sample curve based of the activity of the rhIGF-1/rhIGFBP complex determined in step (d);
   (f) comparing the sample curve to a reference curve indicative of a predetermined activity of the rhIGF-1/rhIGFBP complex to determine the comparability of the sample curve and the reference curve; and
   (g) if the sample curve and the reference curve are not comparable, the lot is not released; if the sample curve and the reference curve are comparable, determining if the lot should be released based on the relative potency of the rhIGF-1/rhIGFBP complex by comparing the sample curve to the reference curve.
2. The lot release assay of paragraph 1, wherein determining the activity of the rhIGF-1/rhIGFBP complex according to step (d) is by measuring the amount of phosphatase produced by the osteosarcoma cells.
3. The lot release assay of paragraph 2, wherein the measuring the amount of phosphatase produced by the osteosarcoma cells is by addition of a substance that reacts with the phosphatase to produce a fluorophore or chromophore, followed by quantifying the amount of the produced fluorophore or chromophore.
4. The lot release assay of paragraph 3, wherein the substance is an aryl phosphate.
5. The lot release assay of paragraph 4, wherein the substance is p-nitrophenylphosphate (PNP).
6. The lot release assay of anyone of paragraphs 2 to 5, wherein the osteosarcoma cells are lysed to release the phosphatase.
7. The lot release assay of anyone of paragraphs 2 to 6, wherein the phosphatase is alkaline phosphatase.
8. The lot release assay of anyone of paragraphs 1 to 7, wherein the rhIGFBP is human IGF-1 Binding Protein 3 (rhIGFBP-3).
9. The lot release assay of anyone of paragraphs 1 to 8, wherein the predetermined period is about 2 days or greater.
10. The lot release assay of paragraph 9, wherein the predetermined period is in the range (for example between) about 2 and 6 days.
11. The lot release assay of paragraph 10, wherein the predetermined period is about 4 days.
12. The lot release assay of any one of paragraphs 1-11, wherein the osteosarcoma cells are derived from an osteosarcoma cell line.
13. The lot release assay of paragraph 12, wherein the osteosarcoma cell line is selected from U-2 OS, MG-63, and Saos-2.
14. The lot release assay of paragraph 13, wherein the osteosarcoma cell line is MG-63.
15. The lot release assay of anyone of paragraphs 1 to 14, wherein lot sample comprising the rhIGF-1/rhIGFBP complex in step (a) is obtained from a drug substance lot.
16. The lot release assay of anyone of paragraphs 1 to 14, wherein lot sample comprising the rhIGF-1/rhIGFBP complex in step (a) is obtained from a drug product lot.
17. The quality control lot release assay of paragraph 16, wherein the lot sample comprising the rhIGF-1/rhIGFBP complex further comprises a surfactant.
18. The lot release assay of paragraph 17, wherein the surfactant is selected from polysorbate 20 (P20) and polysorbate 80 (P80).
19. The lot release assay of paragraph 18, wherein the P20 or the P80 is at a concentration of between about 0.001% (v/v) and about 2.4% (v/v).
20. The lot release assay of paragraph 19, wherein the P20 or the P80 is at a concentration of between about 0.0025% (v/v) and about 0.0075% (v/v).
21. The lot release assay of paragraph 20, wherein the P20 or the P80 is at a concentration of about 0.005% (v/v).
22. The lot release assay of any one of paragraphs 18 to 21, wherein the surfactant is P20.
23. The lot release assay of any one of paragraphs 1 to 22, wherein the contacting the osteosarcoma cells with each of the diluted samples in step (c) is performed in a buffer comprising sodium acetate, acetic acid, and/or sodium chloride.
24. The lot release assay of paragraph 23, the buffer further comprising Triton X-100.
25. The lot release assay of paragraph 24, wherein the Triton X-100 is at a concentration of about 0.05% (v/v) to about 0.15% (v/v).
26. The lot release assay of any one of paragraphs 1 to 25, wherein the series of diluted samples comprising the rhIGF-1/rhIGFB complex generated in step (b) comprise the rhIGF-1/rhIGFBP complex at a concentration of between about 0.3 ng/mL and about 500 ng/mL.
27. The lot release assay of any one of paragraphs 1 to 26, wherein determining if the lot should be released based on the relative potency of the rhIGF-1/rhIGFBP complex in step (g) is by comparing the EC₅₀ of the sample curve to the EC₅₀ of the reference curve, for example to establish if the sample falls within a predefined acceptable range.
28. The lot release assay of paragraph 27, wherein if the EC₅₀ of the sample curve is within about 75% to about 125% of the EC50 of the reference curve the lot is released, for example 75, 80, 85, 90, 91, 92, 93, 94, 95, 96, 97, 98, 99, 100, 101, 102, 103, 104, 105, 106, 107, 108, 109, 110, 115, 120 or 125%.
29. A lot release assay comprising:
   (a) providing a lot sample comprising a recombinantly produced human IGF-1/human IGF-1 Binding Protein (rhIGF-1/rhIGFBP) complex;
   (b) preparing a serial dilution from the lot sample in step (a) to generate a series of diluted samples comprising the rhIGF-1/rhIGFBP complex;
   (c) contacting cells (such as osteosarcoma cells) with each of the diluted samples from step (b) for a predetermined period;
   (d) determining the amount of phosphatase produced by the osteosarcoma cells;
   (e) determining the relative potency of the lot sample by comparing the amount of phosphatase produced by the osteosarcoma cells contacted with each of the dilution samples in step (c) with the amount of phosphatase produced by the osteosarcoma cells contacted with a reference indicative of a predetermined activity of the rhIGF-1/rhIGFBP complex; and
   (f) determining if the lot should be released based on the relative potency of the rhIGF-1/rhIGFBP complex in the lot sample as compared to the reference indicative of a predetermined activity of the rhIGF-1/rhIGFBP complex.
30. The lot release assay of paragraph 29, wherein prior to determining the amount of phosphatase produced by the osteosarcoma cells in step (d) the osteosarcoma cells are lysed.
31. The lot release assay of paragraph 29 or claim 30, wherein the measuring the amount of phosphatase produced by the osteosarcoma cells is by addition of a substance that reacts with the phosphatase to produce a fluorophore or chromophore, followed by quantifying the amount of the produced fluorophore or chromophore.
32. The lot release assay of paragraph 31, wherein the substance is an aryl phosphate.
33. The lot release assay of paragraph 32, wherein the substance is p-nitrophenylphosphate (PNP).
34. The lot release assay of anyone of claims 29 to 33, wherein the phosphatase is alkaline phosphatase.
35. The lot release assay of anyone of paragraphs 29 to 34, wherein the rhIGFBP is human IGF-1 Binding Protein 3 (rhIGFBP-3).
36. The lot release assay of anyone of paragraphs 29 to 35, wherein the predetermined period is about 2 days or greater.
37. The lot release assay of paragraph 36, wherein the predetermined period is in the range (for example between) about 2 and 6 days.
38. The lot release assay of paragraph 37, wherein the predetermined period is about 4 days.
39. The lot release assay of any one of paragraphs 29-38, wherein the osteosarcoma cells are derived from an osteosarcoma cell line.
40. The lot release assay of paragraph 39, wherein the osteosarcoma cell line is selected from U-2 OS, MG-63, and Saos-2.
41. The lot release assay of paragraph 40, wherein the osteosarcoma cell line is MG-63.
42. The lot release assay of anyone of paragraphs 29 to 41, wherein the reference indicative of a predetermined activity of the rhIGF-1/rhIGFBP complex is a reference rhIGF-1/rhIGFBP complex.
43. The lot release assay of paragraph 42, wherein the reference indicative of a predetermined activity of the rhIGF-1/rhIGFBP complex is a reference rhIGF-1/rhIGFBP-3 complex.
44. The lot release assay of anyone of paragraphs 29 to 41, wherein the reference indicative of a predetermined activity of the rhIGF-1/rhIGFBP complex is a reference IGF-1.
45. The lot release assay of paragraph 44, wherein the reference indicative of a predetermined activity of the rhIGF-1/rhIGFBP complex is a reference rhIGF-1.
46. The lot release assay of anyone of paragraphs 29 to 45, wherein lot sample comprising the rhIGF-1/rhIGFBP complex in step (a) is obtained from a drug substance lot.
47. The lot release assay of anyone of paragraphs 29 to 45, wherein lot sample comprising the rhIGF-1/rhIGFBP complex in step (a) is obtained from a drug product lot.
48. The lot release assay of any one of paragraphs 29 to 47, wherein the lot sample comprising the rhIGF-1/rhIGFBP complex further comprises a surfactant.
49. The lot release assay of paragraph 48, wherein the surfactant is selected from polysorbate 20 (P20) and polysorbate 80 (P80).
50. The lot release assay of paragraph 49, wherein the P20 or the P80 is at a concentration of between about 0.001% (v/v) and about 2.4% (v/v).
51. The lot release assay of paragraph 50, wherein the P20 or the P80 is at a concentration of between about 0.0025% (v/v) and about 0.0075% (v/v).
52. The lot release assay of paragraph 51, wherein the P20 or the P80 is at a concentration of about 0.005% (v/v).
53. The lot release assay of any one of paragraphs 29 to 52, wherein the surfactant is P20.
54. The lot release assay of any one of paragraphs 29 to 53, wherein the contacting the osteosarcoma cells with each of the diluted samples in step (c) is performed in a buffer comprising sodium acetate, acetic acid, and/or sodium chloride.
55. The lot release assay of paragraph 54, the buffer further comprising Triton X-100.
56. The lot release assay of paragraph 55, wherein the Triton X-100 is at a concentration of about 0.05% (v/v) to about 0.15% (v/v).
57. The lot release assay of any one of paragraphs 29 to 56, wherein the series of diluted samples comprising the rhIGF-1/rhIGFB complex generated in step (b) comprise the rhIGF-1/rhIGFB complex at a concentration of between about 0.3 ng/mL and about 500 ng/mL.
58. A method of manufacturing a recombinantly produced human IGF-1/human IGF-1 Binding Protein (rhIGF-1/rhIGFBP) complex comprising steps of providing a composition comprising recombinantly produced human IGF-1 (rhIGF-1) and recombinantly produced human IGF-1 Binding Protein (rhIGFBP), for example wherein the rhIGF-1 and rhIGFBP are present at a 1:1 ratio, and wherein the method comprises a lot release assay according to any one of the preceding claims.
59. A drug substance released by a lot release assay according to any one of claims 1 to 57 or produced by a method of manufacture according to claim 58, for example wherein said drug substance has certificate of analysis showing compliance with the release criteria, including us of the same in treatment.
60. A drug product obtained from a drug substance of claim 59.
61. A drug product released by a lot release assay according to any one of claims 1 to 57 or produced by a method of manufacture according to claim 58, for example wherein said drug product has certificate of analysis showing compliance with the release criteria, including use of the same in treatment.
62. Use of an osteosarcoma cell, for example from a cell line (in particular U-2 OS, MG-63, and Saos-2, such as MG-63) for use in a lot release assay according to any one of claims 1 to 57.

In one embodiment the assay according to the present disclosure is a validated process.

Among other things, the present invention provides an accurate and reliable lot release assay that measurement, and ultimately control, for inter-lot variability of the IGF-1 containing composition. Further, the lot release assay controls for variability of the reference standard utilized and assures composition bioavailability. The improvements of the present disclosure are critical for ensuring the quality and safety of IGF-1 containing compositions.

In infants, such as premature infants, minute quantities of the complex are administered as treatment, for example the dose is likely to be in the range of 250 to 500 μg/Kg/day. If the activity of the IGF-1 and/or complex containing same is low then there is a risk that the dose administered will not reach the levels required in vivo. This may mean the infant receive reduced benefit or no benefit from the treatment.

Various aspects of the invention are described in detail in the following sections. The use of sections is not meant to limit the invention. Each section can apply to any aspect of the invention. In this application, the use of “or” means “and/or” unless otherwise stated.

In one aspect, the present invention provides a lot release assay comprising: (a) providing a lot sample comprising a recombinantly produced human IGF-1/human IGF-1 Binding Protein 3 (rhIGF-1/rhIGFBP-3) complex; (b) preparing a serial dilution from the lot sample in step (a) to generate a series of diluted samples comprising the rhIGF-1/rhIGFBP-3 complex; (c) contacting osteosarcoma cells with each of the diluted samples from step (b) for a predetermined period; (d) determining the activity of the rhIGF-1/rhIGFBP-3 complex in the osteosarcoma cells in each of the diluted samples; (e) generating a sample curve based of the activity of the rhIGF-1/IGFBP-3 complex determined in step (d); (f) comparing the sample curve to a reference curve indicative of a predetermined activity of the rhIGF-1/rhIGFBP-3 complex to determine the comparability of the sample curve and the reference curve; and (g) if the sample curve and the reference curve are not comparable, the lot is not released; if the sample curve and the reference curve are comparable, determining if the lot should be released based on the relative potency of the rhIGF-1/rhIGFBP-3 complex by comparing the sample curve to the reference curve.

In another aspect, the present invention provides a lot release assay comprising: (a) providing a lot sample comprising a recombinantly produced human IGF-1/human IGF-1 Binding Protein 3 (rhIGF-1/rhIGFBP-3) complex; (b) preparing a serial dilution from the lot sample in step (a) to generate a series of diluted samples comprising the rhIGF-1/rhIGFBP-3 complex; (c) contacting osteosarcoma cells with each of the diluted samples from step (b) for a predetermined period; (d) determining the amount of phosphatase produced by the osteosarcoma cells; (e) determining the relative potency of the lot sample by comparing the amount of phosphatase produced by the osteosarcoma cells contacted with each of the dilution samples in step (c) with the amount of phosphatase produced by the osteosarcoma cells contacted with a reference indicative of a predetermined activity of the rhIGF-1/rhIGFBP-3 complex; and (f) determining if the lot should be released based on the relative potency of the rhIGF-1/rhIGFBP-3 complex in the lot sample as compared to the reference indicative of a predetermined activity of the rhIGF-1/rhIGFBP-3 complex.

In another aspect, the present invention provides a method of measuring the relative potency of a recombinantly produced human IGF-1/human IGF-1 Binding Protein 3 (rhIGF-1/rhIGFBP-3) complex lot sample, comprising: (a) providing a lot sample comprising a recombinantly produced human IGF-1/human IGF-1 Binding Protein 3 (rhIGF-1/rhIGFBP-3) complex; (b) preparing a serial dilution from the lot sample in step (a) to generate a series of diluted samples comprising the rhIGF-1/rhIGFBP-3 complex; (c) contacting osteosarcoma cells with each of the diluted samples from step (b) for a predetermined period; (d) determining the activity of the rhIGF-1/rhIGFBP-3 complex in the osteosarcoma cells in each of the diluted samples; (e) generating a sample curve based on the activity of the rhIGF-1/IGFBP-3 complex determined in step (d); (f) comparing the sample curve to a reference curve indicative of a predetermined activity of the rhIGF-1/rhIGFBP-3 complex to determine the comparability of the sample curve and the reference curve; and (g) if the sample curve and the reference curve are comparable, determining the relative potency of the lot sample.

In another aspect, the present invention provides a method of manufacturing a recombinantly produced human IGF-1/human IGF-1 Binding Protein 3 (rhIGF-1/rhIGFBP-3) complex comprising steps of providing a composition comprising recombinantly produced human IGF-1 (rhIGF-1) and recombinantly produced human IGF-1 Binding Protein 3 (rhIGFBP-3), wherein the rhIGF-1 and rhIGFBP-3 are present at a 1:1 ratio, and wherein the method of manufacture comprises a lot release assay.

In some embodiments, determining the activity of the rhIGF-1/rhIGFBP-3 complex is by measuring the amount of phosphatase produced by the osteosarcoma cells.

In some embodiments, measuring the amount of phosphatase produced by the osteosarcoma cells is by addition of a substance that reacts with the phosphatase to produce a fluorophore or a chromophore, followed by quantifying the amount of the produced fluorophore or chromophore.

In some embodiments, the substance that reacts with the phosphatase to produce a fluorophore or chromophore is a phosphate derivative. In some embodiments, the substance that reacts with the phosphatase to produce a fluorophore or chromophore is an aryl phosphate. In some embodiments, the substance that reacts with the phosphatase to produce a fluorophore or chromophore is p-nitrophenyl phosphate (PNP).

In some embodiments, the osteosarcoma cells are lysed to release the phosphatase. In some embodiments, the osteosarcoma cells are not lysed.

In some embodiments, the phosphatase is an alkaline phosphatase. In some embodiments, the phosphatase is an acid phosphatase.

In some embodiments, the rhIGFBP is human IGF-1 Binding Protein 3 (rhIGFBP-3).

In some embodiments, the rhIGFBP is human IGF-1 Binding Protein 5 (rhIGFBP-5).

In some embodiments, the predetermined period is in the range (for example between) about 2 and about 6 days. In some embodiments, the predetermined period is between about 3 and about 6 days. In some embodiments, the predetermined period is in the range (for example between) about 2 and about 5 days. In some embodiments, the predetermined period is in the range (for example between) about 3 and about 5 days. In some embodiments, the predetermined period is about 2 days. In some embodiments, the predetermined period is about 3 days. In some embodiments, the predetermined period is about 4 days. In some embodiments, the predetermined period is about 5 days. In some embodiments, the predetermined period is about 6 days.

In some embodiments, the osteosarcoma cells are derived from an osteosarcoma cell line. In some embodiments, the osteosarcoma cell line is selected from is selected from U-2 OS, MG-63, and Saos-2. In some embodiments, the osteosarcoma cell line is U-2 OS. In some embodiments, the osteosarcoma cell line is MG-63. In some embodiments, the osteosarcoma cell line is Saos-2.

In some embodiments, the lot sample comprising the rhIGF-1/rhIGFBP-3 complex is obtained from a drug substance lot. In some embodiments, the lot sample comprising the rhIGF-1/rhIGFBP-3 complex is obtained from a drug product lot.

In some embodiments, the lot sample comprising the rhIGF-1/rhIGFBP-3 further comprises a surfactant. In some embodiments, the surfactant is polysorbate 20 (P20) or polysorbate 80 (P80). In some embodiments, the surfactant is polysorbate 20 (P20).

In some embodiments, the surfactant is at a concentration of between about 0.001% (v/v) and about 2.4% (v/v). In some embodiments, the surfactant is at a concentration of between about 0.001% (v/v) and about 0.01% (v/v). In some embodiments, the surfactant is at a concentration of between about 0.0025% (v/v) and about 0.0075% (v/v). In some embodiments, the surfactant is at a concentration of about 0.0025% (v/v). In some embodiments, the surfactant is at a concentration of about 0.003% (v/v).

In some embodiments, the surfactant is at a concentration of about 0.0035% (v/v). In some embodiments, the surfactant is at a concentration of about 0.004% (v/v). In some embodiments, the surfactant is at a concentration of about 0.0045% (v/v). In some embodiments, the surfactant is at a concentration of about 0.005% (v/v). In some embodiments, the surfactant is at a concentration of about 0.0055% (v/v). In some embodiments, the surfactant is at a concentration of about 0.006% (v/v). In some embodiments, the surfactant is at a concentration of about 0.0065% (v/v). In some embodiments, the surfactant is at a concentration of about 0.007% (v/v). In some embodiments, the surfactant is at a concentration of about 0.0075% (v/v).

In some embodiments, the series of diluted samples comprising the rhIGF-1/rhIGFBP-3 complex comprise the rhIGF-1/rhIGFBP-3 complex at a concentration of between about 0.1 and about 1000 ng/mL. In some embodiments, the rhIGF-1/rhIGFBP-3 complex is at a concentration of between 0.2 ng/mL and about 750 ng/mL. In some embodiments, the rhIGF-1/rhIGFBP-3 complex is at a concentration of between 0.3 ng/mL and about 500 ng/mL.

In some embodiments, the contacting of the osteosarcoma cells with each of the diluted samples is performed in a buffer comprising sodium acetate, acetic acid, and/or sodium chloride.

In some embodiments, the buffer further comprises Triton X-100. In some embodiments the Triton X-100 is at a concentration between about 0.05% (v/v) and about 0.15% (v/v). In some embodiments the Triton X-100 is at a concentration of about 0.05% (v/v). In some embodiments the Triton X-100 is at a concentration of about 0.06% (v/v). In some embodiments the Triton X-100 is at a concentration of about 0.07% (v/v). In some embodiments the Triton X-100 is at a concentration of about 0.08% (v/v). In some embodiments the Triton X-100 is at a concentration of about 0.09% (v/v). In some embodiments the Triton X-100 is at a concentration of about 0.1% (v/v). In some embodiments the Triton X-100 is at a concentration of about 0.11% (v/v). In some embodiments the Triton X-100 is at a concentration of about 0.12% (v/v). In some embodiments the Triton X-100 is at a concentration of about 0.13% (v/v). In some embodiments the Triton X-100 is at a concentration of about 0.14% (v/v). In some embodiments the Triton X-100 is at a concentration of about 0.15% (v/v).

In some embodiments, the series of diluted samples comprising lot samples comprising a rhIGF-1/rhIGFBP-3 complex comprise the rhIGF-1/rhIGFBP-3 complex at a concentration of between about 0.1 ng/mL and about 1000 ng/mL. In some embodiments, the IGF-1/IGFBP-3 complex is at a concentration of between 0.2 ng/mL and about 750 ng/mL. In some embodiments, the IGF-1/IGFBP-3 complex is at a concentration of between 0.3 ng/mL and about 500 ng/mL.

In some embodiments, determining if the lot should be released based on the relative potency of the rhIGF-1/rhIGFBP-3 complex is by comparing the EC₅₀ of the sample curve to the EC₅₀ of the reference curve

In some embodiments, if the EC₅₀ of the sample curve is within about 75% to about 125% of the EC₅₀ of the reference curve the lot is released. In some embodiments, if the EC₅₀ of the sample curve is within about 80% to about 120% of the EC₅₀ of the reference curve the lot is released. In some embodiments, if the EC₅₀ of the sample curve is within about 85% to about 115% of the EC₅₀ of the reference curve the lot is released. In some embodiments, if the EC₅₀ of the sample curve is within about 90% to about 110% of the EC₅₀ of the reference curve the lot is released.

In one embodiment the drug substance according to the present disclosure comprises 45 to 55 μg/ml, for example 50 μg/ml, such as 50 μg/ml+10% overage.

In one embodiment the drug product according to the present disclosure comprises 45 to 55 μg/ml, for example 50 μg/ml, such as 50 μg/ml+10% overage.

### DETAILED DESCRIPTION

The present invention provides, inter alia, an accurate and reliable lot release assay to, inter alia, assess the potency of IGF-1/IGFBP-3 complexes. Additionally, the present invention provides a reliable method for determining the biological activity of an IGF-1/IGFBP-3 complex based on the ability of the complex to stimulate cell growth, proliferation, and/or cAMP production. Further, the present invention provides for manufacturing compositions comprising an insulin-like growth factor-1 (IGF-1), an insulin-like growth factor binding protein 3 (IGFBP-3) and drug substance and/or drug product obtained therefrom.

Drug substance as employed herein refers to GMP manufactured material en route to a final drug product.

Drug product is a final pharmaceutical product suitable for administration to a patient, such as a human. Various aspects of the invention are described in detail in the following sections. The use of sections is not meant to limit the invention. Each section can apply to any aspect of the invention. In this application, the use of “or” means “and/or” unless stated otherwise.

The terms “e.g.” and “i.e.” as used herein, are used merely by way of example, without limitation intended, and should not be construed as referring only those items explicitly enumerated in the specification.

Unless specifically stated or evident from context, as used herein, the term “about” is understood as within a range of normal tolerance in the art, for example within 2 standard deviations of the mean.

“About” can be understood to be within 10%, 9%, 8%, 7%, 6%, 5%, 4%, 3%, 2%, 1%, 0.5%, 0.1%, 0.05%, 0.01%, or 0.001% of the stated value. Unless otherwise clear from the context, all numerical values provided herein reflects normal fluctuations that can be appreciated by a skilled artisan.

### Manufacture of IGF-1 and IGFBP-3

IGF-1 and IGFBP-3 may be produced by a variety of host cells and high expression systems, such as, for example bacteria, yeast, insect, and mammalian cells. Generally, host cells are transformed with DNA expression vectors coding for the IGF-1 or IGFBP-3. Expression vectors containing DNA coding for IGF-1 or IGFBP-3 may be constructed using methods well-known in the art one skilled in the art can adjust and optimize these methods based on common knowledge.

IGF-1 and IGFBP-3 produced by transformed host cells may be purified by a variety of methods known in the art. Poorly soluble proteins may be collected from inclusion bodies following lyses of the host cells and centrifugation. The collected insoluble IGF-1 and/or IGFBP-3 may then be solubilized. Chaotropic solutions (e.g., urea or guanidine containing solutions) may be used for solubilization. Once in solution the IGF-1 and IGFBP-3 may be purified to remove DNA and proteins other than the IGF-1 or IGFBP-3 using a variety of known methods such as precipitation and column chromatography (e.g., hydrophobic interaction chromatography. size exclusion chromatography. ion exchange chromatography, reverse phase high performance liquid chromatography, affinity chromatography, etc.).

Following purification of the crude host cell product, the IGF-1 and IGFBP-3 may be folded into their respective functional conformations by denaturing the protein followed by dilution and reduction. Generally denaturing is accomplished utilizing chaotropic solutions (e.g., solutions containing urea or guanidine). Reduction of IGF-1 and IGFBP-3 may be accomplished concurrently with denaturation or following denaturation. Various agents may be used for reduction, including for example, dithiothreitol (DTT), 2-mercaptoethanol, cysteine, cysteamine, (Z-aminoethanethiol), or reduced glutathione. After refolding is complete, IGF-1 and IGFBP-3 may be purified using a variety of chromatographic methods.

### Lot Release Assay

Lot release as employed herein is the process of evaluating each individual lot of a licensed product before giving approval for its release onto the market. This process is carried out for vaccines and other biologicals in most countries.

Lot release assay as employed herein is an assay (or analytical method) employed in lot release.

The present invention provides methods that may be used to determine the activity of compositions comprising an IGF-1/IGFBP-3 complex. For instance, the present invention provides methods that may be used to determine the activity of lot samples comprising an IGF-1/IGFBP-3 complex. These methods allow for the standardization of the activity for lot samples comprising an IGF-1/IFGBP-3 complex obtained by different methods or sources. The methods also allow for the establishment and assignment of a unit of measurement to facilitate effective and safe administration of composition comprising an IGF-1/IGFBP-3 complex. Further, these methods allow for the determination of whether particular lots comprising an IGF-1/IGFBP-3 (e.g., drug substance lots and drug product lots) meet threshold requirements to be released for distribution and administration to patients.

As used herein, unless the context indicates otherwise, the terms “activity” or “bioactivity” refer to the measure of the biological activity of a sample comprising IGF-1 based on the attributes of IGF-1.

IGF-1 has insulin-like activities and is mitogenic (i.e., stimulates cell division) and/or is tropic (i.e., promotes recovery/survival) for cells in neural, muscular, reproductive, skeletal, and other tissues. Additionally, IGF-1 may paradoxically promote cellular apoptosis or in some instances reduce apoptosis.

IGF-1 exerts its biological activity by binding to the cell surface receptor tyrosine kinases IGF-1 receptor (IGF-1R) and insulin receptor, inducing intracellular signaling cascades. This activity can be measured by quantifying changes in cells contacted with solutions comprising IGF-1. These changes can include, e.g., cell proliferation (e.g., increase in cell number), increased DNA synthesis (e.g., as measured by incorporation of radiolabeled nucleotides (e.g., [³H]thymidine)), and increased or decreased production of certain proteins associated with the cell cycle (e.g., phosphatase).

In one embodiment the lot release may also comprise an analysis of the biological activity of an IGFBP, such as IGFBP-3.

### Lot Sample

As used herein, the terms “lot sample” (also aliquot herein) refers to any specimen of a medium or process solution obtained during the manufacture of an IGF-1/IGFBP-3 complex. A lot sample may be taken or obtained during any operational step or time point of the manufacturing process. In some embodiments, the lot sample is obtained following the recombinant production of a protein. In some embodiments, the lot sample is obtained during the folding or refolding of a protein. In some embodiments, the lot sample is obtained from a process medium prior to filtration. In some embodiments, the lot sample is obtained from a process medium after filtration. In some embodiments, the lot sample is partially purified. In some embodiments, the lot sample is purified. In some embodiments, the lot sample is obtained from a drug substance. In some embodiments, the lot sample is obtained from a formulated drug product.

### IGF-1/IGFBP-3 Complex

As used herein, the terms “IGF-1/IGF Binding Protein 3 complex”, “IGF-1/IGFBP-3 complex”, or simply in some contexts “complex” refer to a noncovalent association generally of equimolar amounts of IGF-1 and an IGFBP-3. The IGF-1/IGFBP-3 complex may be formed by combining approximately equimolar amounts of IGF-1 and IGFBP-3 in a physiologically compatible carrier, e.g., normal saline or phosphate buffered saline solution.

Additionally, the IGF-1/IGFBP-3 complex may be formed by mixing together a concentrated solution of IGF-1 and a concentrated solution of IGFBP-3 for a sufficient time to form an equimolar complex. The IGF-1/IGFBP-3 complex may also be formed during purification as described in WO1996/040736.

It is contemplated that the concentration of the IGF-1/IGFBP-3 complex in a lot sample will vary depending on from which the stage of manufacture that the lot sample is obtained. For example, in some embodiments the concentration of the IGF-1/IGFBP-3 complex is at a concentration of between about 5.0 mg/mL to about 20 mg/mL. In some embodiments the concentration of the IGF-1/IGFBP-3 complex is at a concentration of between about 5.0 [symbol] g/mL to about 500 [symbol] g/mL.

### IGF-1

As used herein, the term “IGF-1” refers to insulin-like growth factor 1. IGF-1 may be naturally occurring and from any species, e.g., bovine, ovine, porcine, equine, and human. IGF-1 may be a variant, analogue, derivative, or mimetic of any naturally occurring IGF-1, whether natural, synthetic, or recombinant, provided that it will bind IGF receptor at the appropriate site. IGF-1 can be produced recombinantly, for example, as described in PCT publication WO95/004076. In some embodiments the IGF-1 is a human IGF-1. In some embodiments, the IGF-1 is recombinantly produced. In some embodiments, the IGF-1 is recombinantly produced human IGF-1 (rhIGF-1).

As used herein, the terms “analogue”, “derivative”, mimetic”, and/or “variant” of IGF-1 are compounds having the same effect as IGF-1 in animals, e.g., humans. These can be naturally occurring analogs of IGF-1 (e.g., truncated IGF-1 (e.g., des(1-3)IGF-1)) or any of the known synthetic analogs of IGF-1. For examples of analogs of IGF-1 see, e.g., U.S. Pat. No. 5,473,054; WO 2011/011071; and WO 2011/011072. IGFBP as employed herein refers to an IGF-1 binding protein, for example selected from IGFBP-1, IGFBP-2, IGFBP-3, IGFBP-4, IGFBP-5, IGFBP-6, IGFBP-7 and combinations of two or more of the same, in particular IGFBP-3 and/or IGFBP-5, more specifically IGFBP-3.

### IGFBP-3

As used herein, the terms “Insulin-like Growth Factor Binding Protein 3” or “IGFBP-3” refers to insulin-like growth factor binding protein 3. IGFBP-3 is a member of the insulin-like growth factor binding protein family. IGFB-3 may be naturally occurring and from any species, e.g., bovine, ovine, porcine, equine, and human. IGFBP-3 may be a variant, analogue, derivative, or mimetic of any naturally occurring IGFBP-3, whether natural, synthetic, or recombinant, provided that it will bind IGF-1 at the appropriate site. IGFBP-3 can be produced recombinantly, as described in PCT publication WO95/004076. In some embodiments, the IGFBP-3 is a human IGFBP-3. In some embodiments, the IGFBP-3 is recombinantly produced. In some embodiments, the IGFBP-3 is recombinantly produced human IGFBP-3 (rhIGFBP-3).

### Buffers and Salts

It is contemplated that a lot sample comprising an IGF-1/IGFBP-3 complex may further comprise a buffer and/or salt.

As used herein, the term “buffer” refers to any pharmaceutically acceptable buffer including, but not limited to, acetate buffer, citrate buffer, phosphate buffer, succinate buffer, and amino acid buffer (e.g., histidine buffer). Suitable buffers may be formed by combining weak acids and/or weak bases with a respective conjugate base or conjugate acid (e.g., acetic acid and sodium acetate). As used herein, the term “salt” refers to any pharmaceutically acceptable salt including, but not limited to sodium chloride and potassium chloride.

In some embodiments, a lot sample comprising an IGF-1/IGFBP-3 complex further comprises a buffer comprising acetate buffer. In some embodiments, the acetate buffer is at a concentration of between about 10 mM to about 100 mM. In some embodiments, the buffer has a pH of between about 5.3 to about 5.8. In some embodiments, the sodium chloride is at a concentration of about 10 mM to about 200 mM.

### Surfactants

It is further contemplated that a lot sample comprising an IGF-1/IGFBP-3 complex may further comprise a surfactant.

As used herein, the term “surfactant” refers to a surface active agent that modifies interfacial tension of water. Typically, surfactants have one lipophilic and one hydrophilic group or region in the molecule. Broadly, the group includes soaps, detergents, emulsifiers, dispersing and wetting agents, and several groups of antiseptics. More specifically, surfactants include stearyltriethanolamine, sodium lauryl sulfate, sodium taurocholate, laurylaminopropionic acid, lecithin, benzalkonium chloride, benzethonium chloride and glycerin monostearate; and hydrophilic polymers such as polyvinyl alcohol, polyvinylpyrrolidone, polyethyleneglycol (PEG), polysorbate (e.g., polysorbate 20 (P20; Tween 20) and polysorbate 80 (P80; Tween 80)), carboxymethylcellulose sodium, methylcellulose, hydroxymethylcellulose, hydroxyethylcellulose and hydroxypropylcellulose or alkyl glycosides.

In some embodiments, a lot sample comprising IGF-1/IGFBP-3 further comprises a surfactant. In some embodiments, the surfactant is polysorbate 20 (P20) or polysorbate 80 (P80). In some embodiments, the surfactant is polysorbate 20 (P20).

In some embodiments, the surfactant is at a concentration of between about 0.001% (v/v) and about 2.4% (v/v). In some embodiments, the surfactant is at a concentration of between about 0.001% (v/v) and about 0.01% (v/v). In some embodiments, the surfactant is at a concentration of between about 0.0025% (v/v) and about 0.0075% (v/v). In some embodiments, the surfactant is at a concentration of about 0.0025% (v/v).

In some embodiments, the surfactant is at a concentration of about 0.003% (v/v). In some embodiments, the surfactant is at a concentration of about 0.0035% (v/v). In some embodiments, the surfactant is at a concentration of about 0.004% (v/v). In some embodiments, the surfactant is at a concentration of about 0.0045% (v/v). In some embodiments, the surfactant is at a concentration of about 0.005% (v/v).

In some embodiments, the surfactant is at a concentration of about 0.0055% (v/v). In some embodiments, the surfactant is at a concentration of about 0.006% (v/v). In some embodiments, the surfactant is at a concentration of about 0.0065% (v/v). In some embodiments, the surfactant is at a concentration of about 0.007% (v/v). In some embodiments, the surfactant is at a concentration of about 0.0075% (v/v).

In some embodiments, a lot sample comprising an IGF-1/IGFBP-3 complex further comprises acetate buffer, sodium chloride, and a surfactant. In some embodiments, a lot sample comprising an IGF-1/IGFBP-3 complex further comprises acetate buffer, sodium chloride, and P20 or P80. In some embodiments, a lot sample comprising an IGF-1/IGFBP-3 complex further comprises acetate buffer, sodium chloride, and P20. In some embodiments, a lot sample comprising an IGF-1/IGFBP-3 complex further comprises acetate buffer, sodium chloride, and P80.

In some embodiments, a lot sample comprising an IGF-1/IGFBP-3 complex further comprises acetate buffer at a concentration between about 10 mM to about 100 mM, sodium chloride at a concentration between about 10 mM to about 200 mM, and P20 at a concentration of about 0.0025% (v/v) and about 0.0075% (v/v), and further wherein the pH is between about 5.3 and about 5.8.

In some embodiments, a lot sample comprising an IGF-1/IGFBP-3 complex further comprises acetate buffer at a concentration of about 50 mM, sodium chloride at a concentration of about 105 mM, and P20 at a concentration of about 0.005% (v/v), and further wherein the pH is about 5.5.

### Cells

As used herein, the term “cell” refers to any cell type that is useful for measuring the activity of an IGF-1/IGFBP-3 complex. Various cell types may be used in the assays of the present invention to assess the activity of the composition comprising the IGF-1/IGFBP-3 complexes.

As discussed briefly above, it is contemplated that IGF-1 exerts its biological activity by binding to IGF-1R and insulin receptor, inducing intracellular signaling cascades. Thus, suitable cell types for the present invention include cells that are sensitive and responsive to changes in the environmental concentration of IGF-1.

Suitable cells may be adherent or non-adherent cell types. Further, this may include primary cells and/or immortalized cell-lines, including those cells that have been used for standard recombinant technology.

In some embodiments, suitable cells may be derived from tissue sources including but not limited to skeletal, cardiac, and smooth muscle; bone; liver; kidney; intestines; skin; nervous system tissues; heart; and lung. Suitable cells may also be epithelial, endothelial, mesenchymal, and neuroectodermal cells.

Particularly suitable cell types for the present invention include osteosarcoma cells. In some embodiments, the osteosarcoma cells are derived from an osteosarcoma cell line. In some embodiments, the osteosarcoma cell line is selected from is selected from U-2 OS, MG-63, and Saos-2.

In some embodiment the osteosarcoma cell line is U-2 OS. In some embodiment the osteosarcoma cell line is MG-63. In some embodiment the osteosarcoma cell line is Saos-2.

### Cell Growth Media

Suitable media for growing cells in culture are well-known in the art. Examples of suitable media are described in “Culture of Animal Cells: A Manual of Basic Technique and Specialized Applications” R. I. Frechney, 2010, Wiley-Blackwell. Typically, optimal medium for each type of cell can be obtained from specialized suppliers. In some embodiments the cells are grown in media comprising RPMI-1640, heat inactivated fetal bovine serum (HI FBS), non-essential amino acid solution (NEAA), and PenStrepGlutamine. In some embodiments the cells are grown in media comprising RPMI-1640, 10% (v/v) HI FBS), 1% (v/v) NEAA, and 1% (v/v) PenSrepGlutamine. Typically, standard cell culture conditions may be used for this purpose. For example, cells may be grown at 37° C. in an incubator containing humidified 95% air/5% CO₂ atmosphere.

### Sample Dilution

It is contemplated that the methods of the present invention comprise preparing a serial dilution from a lot sample comprising an IGF-1/IGFBP-3 complex to generate a series of diluted samples comprising the IGF-1/IGFBP-3 complex.

As used herein, the terms “dilution” or “diluted” refer to any dilution of a substance (e.g., a composition comprising an IGF-1/IGFBP-3 complex) to achieve a desired concentration. In some embodiments, a lot sample may be diluted more than once to achieve multiple diluted samples.

As used herein, the terms “serial dilution” or “serially diluted” refer to a stepwise dilution of a substance resulting in a geometric concentration progression. The dilution factor for a serial dilution may be any factor needed to achieve a desired concentration range for an experiment, e.g., generating a dose-response curve. In some embodiments, a dilution factor may be a ten-fold (10-fold) dilution factor (i.e., a logarithmic dilution), a nine-fold (9-fold) dilution factor, an eight-fold (8-fold) dilution factor, a seven-fold (7-fold) dilution factor, a six-fold (6-fold) dilution factor, a five-fold (5-fold) dilution factor, a four-fold (4-fold) dilution factor, a three-fold (3-fold) dilution factor, a two-fold (2-fold) dilution factor, a one-fold (1-fold) dilution factor, a half-fold (0.5-fold) dilution factor, or a quarter-fold (0.25-fold) dilution factor. In some embodiments, the dilution factor may be a 3.16-fold (i.e., a half-logarithmic dilution) factor. In some embodiments, dilution factor may be a 1.78-fold dilution (i.e., a quarter-logarithmic dilution) factor. In some embodiments, the dilution factor is the same for each dilution step in a serial dilution. In some embodiments, the dilution factor is not the same for each dilution step in a serial dilution.

In one embodiment an aqueous liquid or solution (including a buffer and/or media disclosed herein) is used to dilute the lot.

In some embodiments, diluted lot samples comprising an IGF-1/IGFBP-3 complex comprise the IGF-1/IGFBP-3 complex at a concentration of between about 0.1 ng/mL and about 1000 ng/mL. In some embodiments, the IGF-1/IGFBP-3 complex is at a concentration of between 0.2 ng/mL and about 750 ng/mL. In some embodiments, the IGF-1/IGFBP-3 complex is at a concentration of between 0.3 ng/mL and about 500 ng/mL.

### Contacting Cells

It is contemplated that the methods of the present invention comprise contacting cells with diluted samples comprising an IGF-1/IGFBP-3 complex for a predetermined period. In some embodiments, at least one concentration of an IGF-1/IGFBP-3 complex is tested. In some embodiments, multiple concentrations of the IGF-1/IGFBP-3 complex from the lot sample are tested. In some embodiments, at least two different concentrations of the IGF-1/IGFBP-3 complex are tested. In some embodiments, at least three different concentrations of the IGF-1/IGFBP-3 complex are tested. In some embodiments, at least four different concentrations of the IGF-1/IGFBP-3 complex are tested. In some embodiments, at least five different concentrations of the IGF-1/IGFBP-3 complex are tested. In some embodiments, at least six different concentrations of the IGF-1/IGFBP-3 complex are tested. In some embodiments, at least seven different concentrations of the IGF-1/IGFBP-3 complex are tested. In some embodiments, at least eight different concentrations of the IGF-1/IGFBP-3 complex are tested. In some embodiments, at least nine different concentrations of the IGF-1/IGFBP-3 complex are tested. In some embodiments, at least ten different concentrations of the IGF-1/IGFBP-3 complex are tested. It is contemplated that the multiple concentrations of the IGF-1/IGFBP-3 complex from the lot sample are obtained by serial dilution.

As used herein, the term “predetermined period” refers to any selected amount of time that is sufficient for the pharmacological effects of IGF-1 to affect a cell contacted with solution comprising an IGF-1/IGFBP-3 complex. The predetermined period can be optimized, in relation to the type of cells used in the methods. One skilled in the art can adjust these parameters based on common knowledge.

In one embodiment the predetermined period is 8 hours to 6 days, such as defined elsewhere herein.

### Media for Contacting Cells

Suitable media for contacting the cells with the series of diluted samples comprising an IGF-1/IGFBP-3 complex include suitable growth media for growing cells in culture as discussed above. In some embodiments, the medium used for contacting the cells with the series of diluted samples comprising an IGF-1/IGFBP-3 complex comprises RPMI-1640 medium. In some embodiments, the medium comprises RPMI-1640 medium supplemented with gentamicin, fibronectin, transferrin, ovalbumin, and dexamethasone. In some embodiments, the medium comprises RPMI-1640 medium supplemented with about 0.1 mg/mL Gentamicin, about 2.5 μg/mL Fibronectin, about 4.98 μg/mL Transferrin, about 74.8 μg/mL Ovalbumin, and about 2.98 μM Dexamethasone. Typically, standard cell culture conditions may be used for this purpose. For example, cells may be contacted with the diluted samples of the composition comprising the IGF-1/IGFBP-3 complex at 37° C. in an incubator containing humidified 95% air/5% CO₂ atmosphere for a pre-determined period of time.

### Determining Activity

It is contemplated that contacting cells with diluted IGF-1/IGFBP-3 complex leads to the altered production of various enzymes necessary for growth, proliferation, and apoptosis. This altered production of enzymes (e.g., phosphatase) can be measured by subsequently adding a substrate that an enzyme degrades to a compound the presence of which can be measured by any means known in the art, including spectroscopy or an immunoassay, e.g., Enzyme-linked Immunosorbent Assay (ELISA).

In some embodiments, the present invention comprises determining the activity of a lot sample comprising an IGF-1/IGFBP-3 complex by measuring the amount of phosphatase produced by cells contacted with a series of diluted samples comprising an IGF-1/IGFBP-3 complex for a predetermined period. In some embodiments, the cells are lysed to release phosphatase. In some embodiments, the cells are not lysed.

As used herein, the term “phosphatase” refers to any enzyme that catalyzes the hydrolysis of a phosphoric acid monoester into an alcohol and a phosphate ion. Phosphatases function at a wide array of pH ranges and the term encompasses phosphatases that function optimally below pH 7 (i.e., an acid phosphatase) as well as phosphatases that function optimally above pH 7 (i.e., an alkaline phosphatases).

The amount of phosphatase produced by cells contacted with a series of diluted samples comprising an IGF-1/IGFBP-3 complex may be determined by any means known in the art. Typically, the amount of phosphatase produced by cells may be determined by evaluating the change in fluorescence or absorbance following the addition of a substance (e.g., a fluorogenic compound or a chromogenic compound) that reacts with the enzyme to produce a fluorophore or chromophore. Evaluating the change is fluorescence and/or absorbance may be accomplished by means of suitable equipment, e.g., a spectrophotometer, a spectrofluorometer, a flow cytometer, or a combination thereof.

As used herein, the term “fluorogenic compound” refers to any compound that can be converted by a chemical or biochemical process to a fluorescent compound (i.e., a fluorophore). In some embodiments, the fluorophore is a phosphate derivative.

As used herein, the term “chromogenic compound” refers to any compound that can be converted by a chemical or biochemical process to a colored compound (i.e., a chromophore). In some embodiments, the chromophore is produced from a phosphate derivative. In some embodiments, the chromophore is produced from an aryl phosphate. In some embodiments, the chromophore is produced from a nitrophenyl phosphate. In some embodiments, the chromophore is produced from p-nitrophenyl phosphate.

In some embodiments, the change in fluorescence or absorbance at a particular wavelength indicates an increased production of an enzyme (e.g., phosphatase) by the cells contacted with a series of diluted samples comprising an IGF-1/IGFBP-3 complex for a predetermined period. Thus, in some embodiments, the change in fluorescence or absorbance is correlated with the amount and/or activity of the IGF-1/IGFBP-3 complex in a sample lot. It is contemplated that the absorbance or fluorescence magnitude of the chromophore or fluorophore, respectively, is proportional to the amount of the active enzyme present.

### Assay Buffer

In some embodiments, the measuring the amount of phosphatase produced by the osteosarcoma cells contacted the series of diluted samples is performed in a buffer comprises sodium acetate, acetic acid, and/or sodium chloride.

In some embodiments, the buffer comprises sodium acetate at a concentration of between about 10 mM to about 150 mM. In some embodiment, the pH of the buffer is between about 5.3 and about 5.7.

In some embodiments, the buffer further comprises Triton X-100 (2-[4-(2,4,4-trimethylpentan-2-yl)phenoxy]ethanol). In some embodiments the Triton X-100 is at a concentration between about 0.05% (v/v) and about 0.15% (v/v). In some embodiments the Triton X-100 is at a concentration of about 0.05% (v/v). In some embodiments the Triton X-100 is at a concentration of about 0.06% (v/v). In some embodiments the Triton X-100 is at a concentration of about 0.07% (v/v). In some embodiments the Triton X-100 is at a concentration of about 0.08% (v/v). In some embodiments the Triton X-100 is at a concentration of about 0.09% (v/v). In some embodiments the Triton X-100 is at a concentration of about 0.1% (v/v). In some embodiments the Triton X-100 is at a concentration of about 0.11% (v/v). In some embodiments the Triton X-100 is at a concentration of about 0.12% (v/v). In some embodiments the Triton X-100 is at a concentration of about 0.13% (v/v). In some embodiments the Triton X-100 is at a concentration of about 0.14% (v/v). In some embodiments the Triton X-100 is at a concentration of about 0.15% (v/v).

### Sample Curve

Typically, the activity (e.g., fluorescence or absorbance of each well of the microtiter plate containing different concentrations of IGF-1/IGFBP-3 from a sample lot) is measured and plotted against the corresponding the corresponding concentration of IGF-1/IGFBP-3 to create a dose-response curve. In some embodiments, the dose-response curve is a sigmoidal curve (i.e., “S-shaped”). The range of IGF-1/IGFBP-3 concentrations (e.g., a series of diluted samples) may be expanded to obtain a sigmoidal dose-response curve.

As used herein, the term “sample curve” refers to dose-response curves generated utilizing the series of diluted samples of a lot sample. In some embodiments, one sample curve is generated for a series of diluted samples. In some embodiments, multiple sample curves are generated for a series of diluted samples of a lot sample. In some embodiments, two samples curves are generated for a series of diluted samples of a lot. In some embodiments, three samples curves are generated for a series of diluted samples of a lot sample. In some embodiments, four samples curves are generated for a series of diluted samples.

### Reference Curve

It is further contemplated that in some embodiments, the invention comprises determining the activity of a reference sample.

In some embodiments, a lot sample and a reference sample are tested simultaneously. In some embodiments, a lot sample and a reference sample are tested at different times.

Thus, in one embodiment a reference curve may in fact be a standard set of data stored, for example on an instrument or computer to allow for comparison to the sample (for example where the reference curve is not run at the same time as the reference curve then validation of the comparison needs to be performed and, for example room temperature and/or other physical parameters (such as humidity) may need to be recorded when the assay for the sample is run.

Thus, in one embodiment the reference curve is used to assess multiple sample, for example in one analysis or in multiple analyses. Thus, a stored reference curved may be used for a period of time, for example 1 week, 1 month, 3 months, 6 months or more. This will then be referred to as “the reference curve”.

As used herein, “reference sample” is a sample comprising the same IGF-1/IGFBP-3 complex as the lot sample and further having a known concentration and known activity. This can also be referred to as a STANDARD (in the figures STD1). The reference sample is typically tested at various known concentrations of IGF-1/IGFBP-3. In some embodiments, multiple concentrations of the reference sample may be tested. As with the multiple concentration of the lot sample, the multiple concentrations of the reference sample can be prepared by serial dilution. The concentrations of the IGF-1/IGFBP-3 in the reference sample dilutions may be in the same concentration range as the concentration of the IGF-1/IGFBP-3 in the lot sample dilutions.

The reference sample may be employed to generate a reference curve or “the reference curve”.

For example, in some embodiments, the diluted reference sample comprises the IGF-1/IGFBP-3 complex at a concentration of between about 0.1 ng/mL and about 1000 ng/mL. In some embodiments, the IGF-1/IGFBP-3 complex is at a concentration of between 0.2 ng/mL and about 750 ng/mL. In some embodiments, the IGF-1/IGFBP-3 complex is at a concentration of between 0.3 ng/mL and about 500 ng/mL.

As used herein, the term “reference curve” refers to dose-response curve generated utilizing the series of diluted samples of a reference sample.

### Data Analysis—Comparability

The fluorescence or absorbance readings for each of diluted sample (e.g., the series of diluted samples of a lot sample or a reference standard) comprising an IGF-1/IGFBP-3 complex, known as raw data, can be processed and subject to further statistical analysis. Dedicated software can be employed for statistical analysis, for example PLA3 (Stegmann Systems GmbH, Germany) or, alternatively, a commercial off-the-shelf spreadsheet customized for statistical evaluation of biological assay data.

It is contemplated that the differences between a sample curve and a reference curve is a function of the difference in the biological activity between the lot sample and the reference sample.

In some embodiments, a four-parameter logistic function model is used to determine the differences between the sample curve and the reference curve. In some embodiments a linear function model is used to determine the differences between the samples curve and the reverse curve. In some embodiments, a higher-order polynomial function is used to determine the difference between the sample curve and the reference curve. In some embodiments, a five-parameter logistic function model is used to determine the differences between the sample curve and the reference curve. In some embodiments, the data obtained from the activity measurements (e.g., a sample curve and/or a reference curve) can be evaluated for additional statistical parameters to ensure that the data are valid.

In some embodiments, the sample curve and the reference curve are compared to determine if the respective dose-response curves are comparable.

As used herein, the terms “comparable”, and conversely “not comparable”, as used to describe the relationship of a sample curve and reference curve and refer to the similarity of the curves. The similarity of a sample curve and a reference curve may be described by various relationships (e.g., similarity between the upper asymptote, the lower asymptote, and/or the slope of the curve). A sample curve is considered comparable to a reference curve if certain measures are within defined parameters. In some embodiments, a sample curve is considered comparable to a reference curve when the coefficient of determination (R²) is greater than or equal to 0.95, the curve amplitude (A/D) ratio is between 0.396 and 1.727, the ratio of difference in asymptotes (A−D Ratio) is between 0.701 and 1.357, the ratio of slopes (B Ratio) is between 0.377 and 1.650, and/or the aggregate value of geometric coefficient of variance (% GCV) is less than or equal to 25% from multiple curve comparisons. In some embodiments a sample curve is considered comparable to a reference curve when the coefficient of determination (R²) is greater than or equal to 0.95. In some embodiments, a sample curve is considered comparable to a reference curve when the curve amplitude (A/D) ratio is between 0.396 and 1.727. In some embodiments, a sample curve is considered comparable to a reference curve when the ratio of difference in asymptotes (A−D Ratio) is between 0.701 and 1.357. In some embodiments, a sample curve is considered comparable to a reference curve when the ratio of slopes (B Ratio) is between 0.377 and 1.650. In some embodiments, a sample curve is considered comparable to a reference curve when the aggregate value of geometric coefficient of variance (% GCV) is less than or equal to 25% from multiple curve comparisons. Conversely, a sample curve is not considered comparable to reference curve if the above criteria are not met.

As used herein, the term “curve amplitude” refers to the ratio between the upper asymptote (A) and lower asymptote (D) of a dose response curve:

$$Curve Amplitude = \frac{A}{D}$$

As used herein, the term “difference in asymptotes” refers to the difference between the upper asymptote (A) of a dose response curve and the lower asymptote (D) of a dose response curve:

Difference in Asymptotes=A−D

As used herein, the term “curve amplitude ratio” or “A/D Ratio” refers to the ratio of the curve amplitude of a sample dose response curve and a reference dose response curve:

$$\frac{A}{D} Ratio = \frac{{( A / D )}_{sample}}{{( A / D )}_{Reference}}$$

As used herein, the term “ratio of difference in asymptotes” or “A−D Ratio” refers to the difference in asymptotes of a sample dose response curve to the difference in asymptotes of a reference dose response curve:

$$Ratio of Difference in Asymptotes = \frac{{( A - D )}_{Sample}}{{( A - D )}_{Reference}}$$

As used herein, the phrase “ratio of hill slopes” or “B Ratio” refers to the ratio of the slope of a reference dose response curve and the slope of a sample dose response curve:

$$Ratio of Hill Slopes = \frac{{B}_{Reference}}{{B}_{Sample}}$$

It is contemplated that if a sample curve and a reference curve are not comparable, the lot from which the lot sample was obtained is not released and is further evaluated.

### Relative Potency

It is contemplated that if a sample curve and a reference curve are determined to be comparable, determining whether the lot should be released is based on the relative potency of the IGF-1/rhIGFBP-3 complex by comparing the sample curve to the reference curve.

In some embodiments, the present invention comprises comparing the activity measured for lot samples containing an IGF-1/IGFBP-3 complex to the activity measured for reference standards containing the IGF-1/IGFBP-3. Thus, the relative potency of the sample lot IGF-1/IGFBP-3 complex can be calculated based on the activity measurements from the lot sample by comparison to a standard calibration curve obtained from the activity analysis of the reference standard or a population of lot samples.

In some embodiments, the activity measurements for the lot sample and the reference standard are acquired at the same time. In other embodiments, the activity measurements for the lot sample and the reference standard are obtained at different times.

As used herein, the term “relative potency” refers a measure of activity of a lot sample comprising IGF-1/IGFBP-3 relative to a reference standard of IGF-1/IGFBP-3 having a known activity. For example, the relative potency may be expressed as the ratio of the half maximal effective concentration (EC₅₀) of the reference curve to the EC₅₀ of the sample curve.

$$Relative Potency = \frac{E {C}_{50} Reference}{E {C}_{50} Sample}$$

For clarity, determining relative potency requires that the EC₅₀ values are derived from the restricted fits after evaluating the comparability/equivalency of the sample and reference curves.

In some embodiments, the relative potency is expressed as a percentage. In some embodiments, the relative potency of the lot sample and a reference standard are equal (i.e., 100%). In some embodiments the relative potency of a lot sample is determined to be identical to the reference standard when the comparison between the lot sample activity and the reference standard activity fall within an acceptable range. In some embodiments, the acceptable range is about 75% to about 125%, about 80% to about 120%, or about 90% to about 110%.

Values and/or features in an Example(s) may be used as basis for amendment to the claims, wherein the value and/or feature is employed without reference to other elements used in the Example.

“Is” as employed herein means comprising.

In the context of this specification “comprising” is to be interpreted as “including”.

Embodiments of the invention comprising certain features/elements are also intended to extend to alternative embodiments “consisting” or “consisting essentially” of the relevant elements/features.

Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

The background section contains technically relevant details may be used as basis for an amendment.

### BRIEF DESCRIPTION OF THE DRAWING

The accompanying drawings, which are incorporated in and constitute a part of this specification, illustrate embodiments of the invention and, together with the description, serve to explain the objects, advantages, and principles of the invention. Drawings are for illustrative purposes only and not for limitation.

FIG. 1 depicts a general schematic of a disclosed lot release assay.

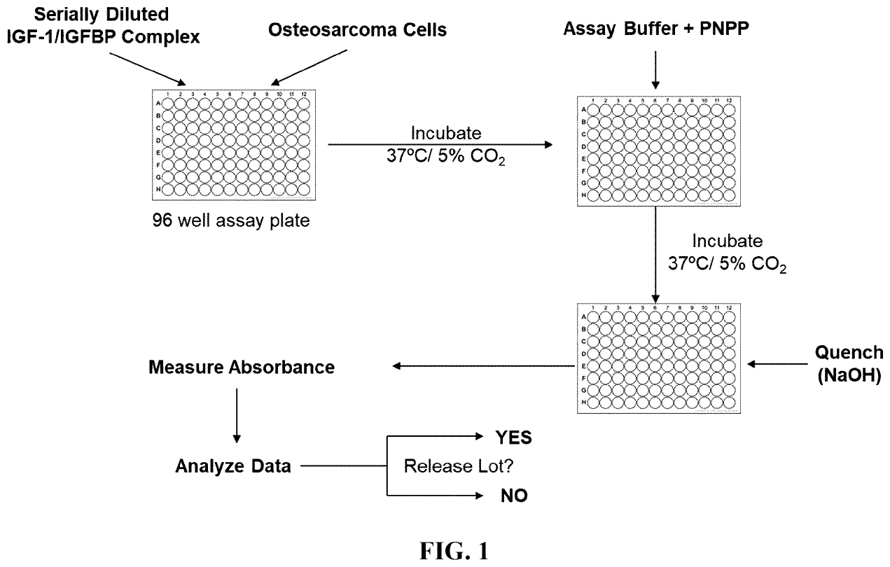

FIG. 2 depicts a reference curve and a sample curve for a composition comprising rhIGF1-/rhIGFBP-3.

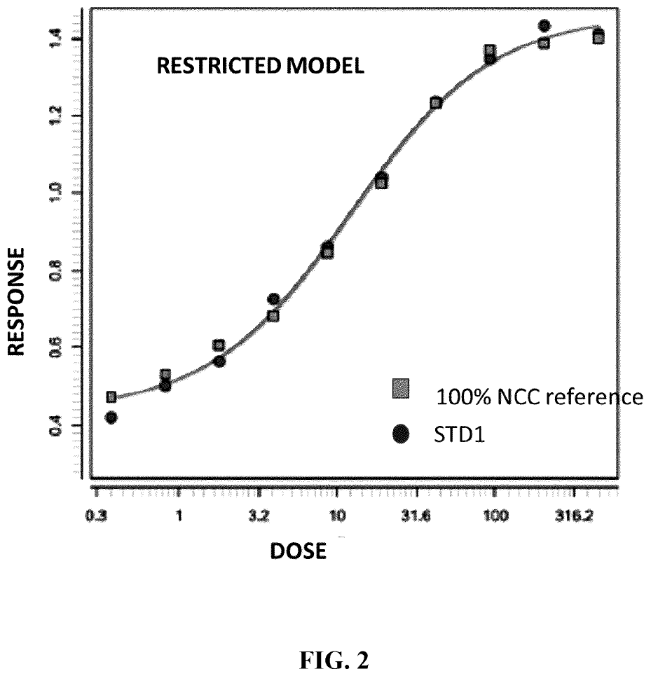

FIG. 3 depicts a reference curve and a sample curve for a composition comprising rhIGF1-/rhIGFBP-3 having approximately 30% degradation

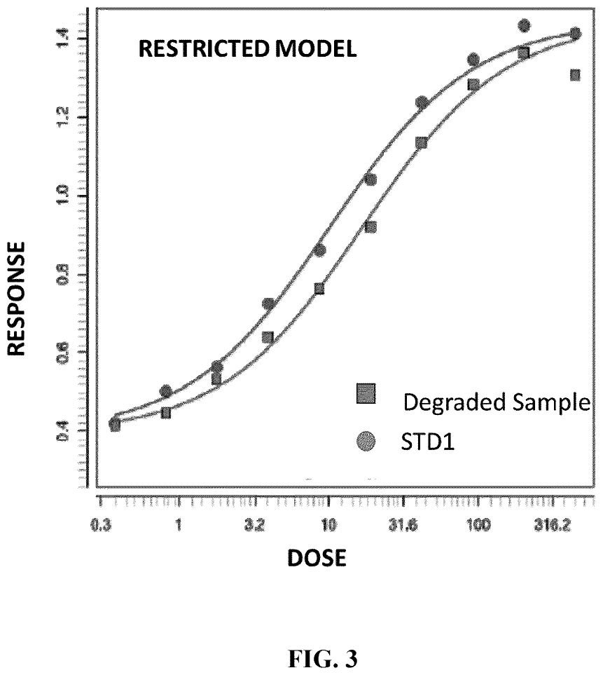

FIG. 4 depicts a reference curve and a sample curve for a composition comprising rhIGF1-/rhIGFBP-3 a fully degraded rhIGF-1/IGFBP-3 composition.

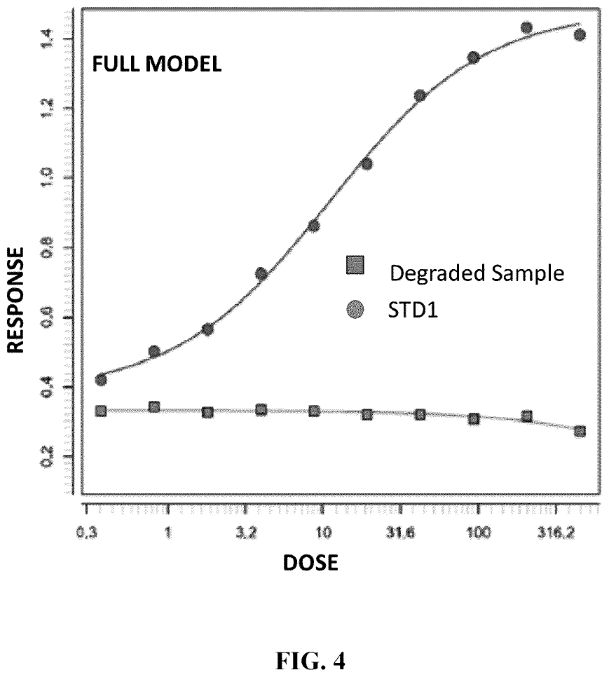

### EXAMPLE 1

While certain compounds, compositions and methods described herein have been described with specificity in accordance with certain embodiments, the following examples serve only to illustrate the compounds of the invention and are not intended to limit the same.

### Reagent Preparation:

MG-63 Growth Media. RPMI-1640 (880 mL), heat inactivated fetal bovine serum (FBS) (100 mL), PenStrepGlutamine (100×; 10 mL), and non-essential amino acids (NEAA) (100×; 10 mL) were combined, mixed, and sterile filtered. Final concentrations were 10% FBS, 1% NEAA, and 1% PenStrepGlutamine.

5× Assay Media. RPMI-1640 (880 mL), gentamicin (10 mL of 50 mg/mL), fibronectin (12.5 mL of 1 mg/mL), Transferrin (10 mL 2.5 mg/mL), ovalbumin (10 mL of 37.5 mg/mL), and dexamethasone (80 mL of 187 μM) were combined, mixed, and sterile filtered. Final concentrations were 0.5 mg/mL Gentamicin, 12.5 μg/mL Fibronectin, 24.9 μg/mL Transferrin, 374 μg/mL Ovalbumin, and 14.9 μM Dexamethasone.

1× Assay Media. RPMI-1640 (800 mL), 5× Assay Medium (200 mL), NEAA (100×; 10 mL) of, and L-Glutamine (100×; 10 mL) were combined, mixed, and sterile filtered.

Sodium Acetate Buffer. Anhydrous sodium acetate (8.2±0.4 g) was dissolved in cell certified water (900 mL) and Triton X-100 (1 mL) was added. Total volume was brought to 1 L and HCl (6N) was used to adjust the pH as needed with a target of pH 5.5±0.1.

Drug Substance Formulation Buffer. Sodium acetate trihydrate (5.92±0.01 g), sodium chloride (6.14±0.02 g), and glacial acetic acid (0.35±0.01 g) were added to cell certified water (800 mL). The solution was mixed and brought to a total volume of 1 L and pH was adjusted as needed with a target of pH 5.5±0.1.

One Percent (1%) Polysorbate 20 (P20) Stock Solution. Polysorbate 20 (0.11±0.02 g) was dissolved in Drug Substance Formulation Buffer (10 mL) mixed and sterile filtered.

1× Working Assay Buffer (WAB). P20 stock solution (1 mL) was with 250 mL of 1× assay buffer.

p-Nitrophenyl Phosphate (PNP) Stock. para-Nitrophenyl Phosphate hexahydrate (0.975 to 1.002 g) was dissolved in Sodium Acetate Buffer (7.4 mL) and mixed. Final concentration of PNP was 0.36 M.

Working PNP Solution. PNP stock solution (1 mL) was added to sodium acetate buffer (49 mL). Final PNP concentration was 7.2 mM.

Dexamethasone Stock. Dexamethasone (29.5 mg) was dissolved in absolute ethanol and mixed. RPMI-1640 (400 mL) was added and the solution was mixed and filtered. Final concentration of dexamethasone was 187 M.

Transferrin Stock. Transferrin (500 mg) was dissolved in RPMI-1640 (200 mL). The solution was mixed and filtered. Final transferrin concentration was 2.5 mg/mL.

Ovalbumin Stock. Ovalbumin (1.5 g) was added to RMPI-1640 (40 mL) mixed and filtered. Final concentration of ovalbumin was 37.5 mg/mL.

### Cell Culture

MG-63 cells were maintained in T225 flasks in growth media. Culture flasks were removed from incubator to confirm that confluency was between 70-95% after which growth media was aspirated and the cells were washed with versene (about 10 mL), which was then removed by aspiration. Additional versene (2-3 mL) was added to the culture flasks which were then incubated with gentle rocking at 37° C., 5% CO₂ for about 5 minutes.

Once the cells were dislodged, 1× assay buffer (8-12 mL) was added to each flask and the cells were gently mixed by pipetting. The resulting cell suspensions were pooled as necessary, transferred to sterile tubes, and pelleted by centrifugation (about 200 g for 5 minutes). Supernatant was aspirated and the cell pellets were resuspended in 1× assay buffer (10 mL per flask). Cells were counted and then diluted to achieve a final suspension of about 0.1×10⁶ cells/mL. Each assay requires four 96-well plates and 45 mL of cell suspension. Cells used in the assay should be >95 but ≤111 (i.e., cell passage number).

### Sample Preparation

Vials of reference standard (reference), assay control, (control) and test sample (sample) were removed from the −80° C. freezer and thawed on ice. Reference standard and assay control (10 μL each) were diluted in 990 μL of WAB to achieve a concentration of 100 μg/mL (pre-dilution 1). Pre-dilution 1 (100 μL each) was further diluted with 900 μL of WAB to achieve a concentration of 10 μg/mL (pre-dilution 2). Pre-dilution 2 (90 μL each) was further diluted with 910 μL WAB (1×) to achieve a concentration of 900 ng/mL. Test samples were tested at 100% nominal drug concentration (NDC). The desired final sample volume required for one assay is 1 mL and the target sample concentration is 0.9 μg/mL. Four replicates for each reference, control, and sample were prepared.

### Assay

Four 96-well assay plates were filled with 180 μL of WAB per well, except for column 2, rows B-G which were left empty for references, controls, and lot samples. Column 2, rows B-G, of each plates were filled with 330 μL of the final dilutions for each reference, control, and sample as show in Table 1, below.

| | MG-63 Proliferation Assay Plate Arrangement | | | |
|---|---|---|---|---|
| Row | Plate 1 | Plate 2 | Plate 3 | Plate 4 |
| B | Sample 1a | Sample 2b | Sample 3c | Control d |
| C | Reference 1a | Reference 1b | Reference 1c | Reference 1d |
| D | Sample 2a | Sample 3b | Control c | Sample 1d |
| E | Sample 3a | Control b | Sample 1c | Sample 2d |
| F | Reference 2a | Reference 2b | Reference 2c | Reference 2d |
| G | Control a | Sample 1b | Sample 2c | Sample 3d |

Each reference, control, and sample was serially diluted by transferring 150 μL from column 2 to column 3 of each assay plate, and so on to column 12.

Prepared cell suspension (100 μL) was added to each well of 96-well assay plates. Assay plates were then covered and placed in a humidified incubator at 37° C., 5% CO₂ for 72±3 hours (i.e., 3 days).

### Detection

Plates were removed from incubator and washed with Dulbecco's Phosphate Buffered Saline (DPBS). Plates were then blotted with absorbent towels to remove excess liquid. Working PNP solution was added to the wells (100 μL each), taking care to avoid bubbles. Plates were covered and incubated at 37° C. for about 120-130 minutes. The reaction was then quenched by addition of aqueous NaOH (50 μL; 0.5N) to each well. The plates were again covered and incubated at room temperature for 10-60 minutes before reading the absorbance at 405 nm and 490 nm using a Synergy Neo2 (BioTek Instruments, Winooski, VT).

### Data Capture and Analysis

Raw data was transformed by subtracting the raw 490 nm absorbance data from the raw 405 nm absorbance data, followed by importing into PLA 3.0 (GEN5) for analysis. The reference, assay control, and sample data points were fitted to a 4-parameter logistic (4-PL) curve fit:

$$Y = \frac{A - D}{1 + {( \frac{X}{C} )}^{B}} + D$$

A=absorbance at concentration 0 (upper D=the absorbance at infinite concentration asymptote) (lower asymptote)
B=the measure of the hill curve (i.e., steepness) X=concentration of IGF-1/IGFBP-3 complex
C=the value of X at the inflection point (i.e., the Y=transformed absorbance signal EC50)

### Assay Acceptance Criteria

A given assay was considered acceptable if the following conditions were satisfied:

R²≥0.95
A/D Ratio=1.177 to 5.735
A−D Ratio=0.108 to 2.412

If a reference replicate fails to meet criteria, all remaining data in comparison to that reference replicate is excluded.

Further, each control replicate should meet the following criteria:

R²≥0.95
A/D Ratio=0.791 to 1.279 A−D Ratio=0.843 to 1.189 B Ratio=0.600 to 1.360

If control replicate fails to meet criteria, all remaining data in comparison to that control replicate and reference replicate combination is excluded.

If the reference and control curves meet their respective acceptance criteria, then the sample curve is accessed for comparability. A sample curve is considered comparable if the following criteria are satisfied:

R²≥0.95
A/D Ratio=0.396 to 1.727
A−D Ratio=0.701 to 1.357
B Ratio=0.377 to 1.650

Additionally, the % GCV should be ≤25% for the aggregated data.

### Sample Curve Comparability

If the sample curve replicate fails to meet criteria, it is excluded from final % GCV and reportable value calculations. A minimum of n=3 across the four-plate assay must be met to generate the final reportable value. Test outliers are also excluded from data analysis.

1-44. (canceled)

45. A lot release assay method for determining whether to release a lot in order tor ensure activity of IGF-1 is suitable for treatment of prevention of conditions resulting from deficiency of IGF-1 (for example in premature infants) comprising:
(a) providing a lot sample comprising:
a recombinantly produced human IGF-1/human IGF-1 Binding Protein-3 (rhIGF-1/rhIGFBP) complex at 45 to 55 μg/ml,
a surfactant selected from polysorbate 20 and polysorbate 80 at a concentration between about 0.0025% v/v and about 0.0075% v/v;
(b) preparing a serial dilution from the lot sample in step (a) to generate a series of diluted samples comprising the rhIGF-1/rhIGFBP complex;
(c) contacting osteosarcoma cells with each of the diluted samples from step (b) for a predetermined period;
(d) determining the activity of the rhIGF-1/rhIGFBP complex in the osteosarcoma cells in each of the diluted samples;
(e) generating a sample curve based of the activity of the rhIGF-1/rhIGFBP complex determined in step (d);
(f) comparing the sample curve to a reference curve indicative of a predetermined activity of the rhIGF-1/rhIGFBP complex to determine the comparability of the sample curve and the reference curve; and
(g) if the sample curve and the reference curve are not comparable, the lot is not released; if the sample curve and the reference curve are comparable, determining if the lot should be released based on the relative potency of the rhIGF-1/rhIGFBP complex by comparing the sample curve to the reference curve.

46. A lot release assay method for determining whether to release a lot in order to ensure activity of IGF-1 is suitable for treatment of prevention of conditions resulting from deficiency of IGF-1 (for example in premature infants) comprising:
(a) providing a lot sample comprising:
a recombinantly produced human IGF-1/human IGF-1 Binding Protein-3 complex at 45 to 55 μg/ml,
a surfactant selected from polysorbate 20 and polysorbate 80 at a concentration between about 0.0025% v/v and about 0.0075% v/v;
(b) preparing a serial dilution from the lot sample in step (a) to generate a series of diluted samples comprising the rhIGF-1/rhIGFBP complex;
(c) contacting osteosarcoma cells with each of the diluted samples from step (b) for a predetermined period;
(d) determining the amount of phosphatase produced by the osteosarcoma cells;
(e) determining the relative potency of the lot sample by comparing the amount of phosphatase produced by the osteosarcoma cells contacted with each of the dilution samples in step (c) with the amount of phosphatase produced by the osteosarcoma cells contacted with a reference indicative of a predetermined activity of the rhIGF-1/rhIGFBP complex; and
(f) determining if the lot should be released based on the relative potency of the rhIGF-1/rhIGFBP complex in the lot sample as compared to the reference indicative of a predetermined activity of the rhIGF-1/rhIGFBP complex.

47. The lot release assay of claim 45, wherein determining the activity of the rhIGF-1/rhIGFBP complex according to step (d) is by measuring the amount of phosphatase produced by the osteosarcoma cells.

48. The lot release assay of claim 45, wherein the measuring the amount of phosphatase produced by the osteosarcoma cells is by addition of a substance that reacts with the phosphatase to produce a fluorophore or chromophore, followed by quantifying the amount of the produced fluorophore or chromophore.

49. The lot release assay of claim 48, wherein the substance is an aryl phosphate, such as p-nitrophenylphosphate (PNP)

50. The lot release assay of claim 45, wherein the osteosarcoma cells are lysed to release the phosphatase.

51. The lot release assay of claim 45, wherein the predetermined period is about 2 days or greater, for example wherein the predetermined period is in the range about 2 and 6 days.

52. The lot release assay of claim 51, wherein the predetermined period is about 4 days.

53. The lot release assay of claim 45, wherein the osteosarcoma cells are derived from an osteosarcoma cell line, for example selected from U-2 OS, MG-63, and Saos-2, such as MG-63.

54. The lot release assay of claim 45, wherein the contacting the osteosarcoma cells with each of the diluted samples in step (c) is performed in a buffer comprising sodium acetate, acetic acid, and/or sodium chloride.

55. The lot release assay of claim 45, wherein determining if the lot should be released based on the relative potency of the rhIGF-1/rhIGFBP complex in step (g) is by comparing the EC50 of the sample curve to the EC50 of the reference curve.

56. The lot release assay of claim 55, wherein if the EC50 of the sample curve is within about 90% to about 110% of the EC50 of the reference curve the lot is released.

57. The lot release assay according to claim 45, wherein the following criteria are satisfied:
R²≥0.95,
A/D Ratio is in the range 1.1177 to 5.735
A−D Ratio is in the range 0.108 to 2.412, and
where a reference replicate fails to meet said criteria, all remaining data in comparison to that reference replicate is excluded, and
a sample curved is considered comparable if the following criteria are satisfied:
R²≥0.95,
A/D Ratio is in the range 0.396 to 1.727
A−D Ratio is in the range 0.701 to 1.357
B Ratio is in the range 0.377 to 1.650,
the % GCV should be ≤25% for the aggregated data, and
a minimum of n=3 across a four-plate assay must be met to generate the final reportable value.

58. A method of manufacturing a recombinantly produced human IGF-1/human IGF-1 Binding Protein (rhIGF-1/rhIGFBP) complex lot for release comprising the steps of:
a) providing a composition comprising recombinantly produced human IGF-1 (rhIGF-1) and recombinantly produced human IGF-1 Binding Protein-3 at 45 to 55 μg/ml, a surfactant selected from polysorbate 20 and polysorbate 80 at a concentration between about 0.0025% v/v and about 0.0075% v/v, for example wherein the rhIGF-1 and rhIGFBP are present at a 1:1 ratio, and
b) performing a lot release assay according to claim 45, and
c) generating a certificate of analysis showing compliance with release criteria.

59. A method of manufacturing a recombinantly produced human IGF-1/human IGF-1 Binding Protein (rhIGF-1/rhIGFBP) complex lot for release comprising the steps of:
a) providing a composition comprising recombinantly produced human IGF-1 (rhIGF-1) and recombinantly produced human IGF-1 Binding Protein-3 at 45 to 55 μg/ml, a surfactant selected from polysorbate 20 and polysorbate 80 at a concentration between about 0.0025% v/v and about 0.0075% v/v, for example wherein the rhIGF-1 and rhIGFBP are present at a 1:1 ratio, and
b) performing a lot release assay according to claim 46, and
c) generating a certificate of analysis showing compliance with release criteria.

60. A drug product obtained from the method of claim 58.

61. A drug product obtained from the method of claim 59.
